(19) United States
(12) Patent Application Publication
(10) Pub. No.: US 2026/0226522 A1
(43) Pub. Date: Aug. 6, 2026

(54) CARBOHYDRATE COMPOSITION AND METHODS FOR PRODUCING ALPHA-GLUCOOLIGOSACCHARIDES AND USE THEREOF

(71) Applicant: BIONEUTRA NORTH AMERICA INC., Edmonton (CA)

(72) Inventors: Ying HU, Edmonton (CA); Vinti GOEL, Edmonton (CA); Gonglai YAN, Edmonton (CA); Jianhua ZHU, Edmonton (CA)

(21) Appl. No.: 19/149,442

(22) PCT Filed: Jan. 22, 2024
(86) PCT No.: PCT/CA2024/050068

§ 371 Date: Jul. 18, 2025

Related U.S. Application Data
(60) Provisional application No. 63/440,337, filed on Jan. 20, 2023.

(51) Int. Cl. C12P 19/04; C07H 3/06; C12P 19/14; C12P 19/18

(52) U.S. Cl. CPC C12P 19/04; C07H 3/06; C12P 19/14; C12P 19/18

(57) ABSTRACT

A method for preparing alpha-glucooligosaccharides (AGO) from starch employs simultaneous or sequential saccharification and multiple transfer reactions with two or more commercial glycosyltransferases, including Glyco Transferase, starch branching enzyme, cyclodextrin glycosyltransferases, and transglucosidase. Relative to common types of IMO, the chemical structure of AGO may be characterized by increased oligosaccharides with alpha-1,6 glycosidic linkages, or newly synthesized alpha-1,3 glycosidic linkages, or cyclic-alpha-1,4 glycosidic linkages with a medium dextrose equivalent (DE) between 20 and 30 to enhance the fiber content.

### TECHNICAL FIELD

This relates to a method of producing alpha-glucooligosaccharides (AGO), and in particular, producing AGO from starch by enzymatic modification.

### BACKGROUND

Polysaccharides such as cellulose, starch, or chitin are the most abundant renewable resources on earth. Starch serves as not only the most dominant energy storage carbohydrate primarily synthesized through plant photosynthesis but also a major constituent source of calories in the human diet released through the starch digestion process. Natural starches generally consist of about 10-30% amylose and 70-90% amylopectin. However, the ratio may vary largely so that the proportion of amylose may range from nearly undetectable to 70%. Starch does not taste sweet before it breaks down into glucose, maltose, or oligosaccharides. In addition to cereals or tubers containing starch as food and feed, starch from harvested grains and tubers is also used for enzymatic hydrolysis and industrial fermentation to manufacture numerous products by a wide variety of enzymes.

Enzymes possess remarkable specificity and high selectivity in the biocatalytic conversion of only one type or similar types of substrate molecules into product molecules under mild conditions. In this context, the biocatalytic strategy has several advantages over chemical synthesis reactions as a greener process and safe and scalable biocatalytic route. Many enzymes are widely used in various industries, especially food processing. Many enzymes from microbial origins are commercially available; their stability makes them more suitable for industrial processing, such as thermal-stable alpha-amylases from thermophilic microorganisms with high-temperature stability, which is primarily used within starch processing. Commonly used starch-modifying enzymes include alpha-amylase, beta-amylase, glucoamylase, pullulanase, and alpha-glucosidase of microbial origin, which are utilized in a wide variety of industrial applications. They benefit from many biocatalytic routes to obtain starch-derived oligosaccharides by modifying the linkage types to change the health benefits.

Starch and starch-derived polysaccharides and oligosaccharides must be hydrolyzed to their constituent monosaccharides before being absorbed by the body. Dietary fiber has long been recommended as part of a healthy diet. High levels of foods containing fiber have decreased the risk of a variety of chronic health disorders such as cardiovascular disease, type II diabetes, and certain cancers. The FDA recommends total dietary fiber intake at about 25 g/day, of which about 25% (about 6 g) should be soluble fiber. Thus, there is a need to increase fiber consumption, and many novel isolated or synthesized fibers may easily be added to beverages and processed foods. Starch is classified into three nutritional types: rapidly digestible starch, slowly digestible starch, and resistant starch. However, resistant starch is an insoluble fiber. Non-digestible oligosaccharides with better solubility have drawn recent interest, such as resistant dextrin (RD). Fibersol-2®, a water-soluble fiber product, is produced by chemical pyrolysis and controlled enzymatic hydrolysis of cornstarch by alpha-amylase as published in U.S. Pat. Nos. 5,620,873 and 5,358,729. It has an average molecular weight of 2000 Da with a random distribution of alpha- and beta-(1,4), (1,6), (1,2), and (1,3) linkages. The fiber content of Fibersol-2® is around 90%, which may escape digestion in the upper gastrointestinal tract and reach the colon. However, only half is in vivo fermented by the microbiota, resulting in significantly lower total tract carbohydrate and glucose digestibility values.

Oligosaccharide digestion in the small intestine relies on pancreatic amylases and membrane-bound brush border enzymes, the most important of which are maltase, sucrase, isomaltase, and lactase. Commercial isomalto-oligosaccharides (IMO) product is an emerging prebiotic, a mixture of slow-digestible oligosaccharides formed by glucose residues mainly linked by alpha-(1,6) linkages and next by alpha-(1,4) linkages, and a much lower extent of alpha-(1,2) and alpha-(1,3) linkages, including isomaltose, panose, isomaltotriose, isomaltopentaose and various branched oligosaccharides. It is known that alpha-(1,6) linkages are hydrolyzed by the mucosal alpha-glucosidases in the small intestine but at a lower rate than alpha-(1,4) linkages. Alpha-(1,2) and alpha-(1,3) linkages are regarded as non-digestible linkages. In addition, IMO and oligodextrans may also be synthesized from sucrose using the enzymes dextransucrase and dextranase. Currently, IMO have been enzymatically produced from a wide range of starches via an enzymatic process. The reactions are influenced by the ratio of amylose and amylopectin in different crops, including corn, wheat, barley, potato, pea, tapioca, etc. A commercially scaled starch-derived process may involve a three-step procedure of liquefaction, saccharification, and transglycosylation. First, the starch is hydrolyzed to produce dextrin or maltodextrin using liquefaction enzymes, such as thermal-stable alpha-amylase. In the next step, the dextrin/maltodextrin solution is saccharified by beta-amylase or fungal alpha-amylase into maltose and oligomers with low degrees of polymerization (DPs) with or without debranching enzyme (pullulanase) to obtain higher maltose and maltotriose solution and residual short maltodextrins with predominant alpha-(1,4) glycosidic linkages. In the transglycosylation step, the transglucosidase from Aspergillus niger, an alpha-glucosidase that is normally used for hydrolysis of maltose and oligomers with low DPs but also transfers the glucosyl moiety from donor molecules to the acceptor molecules with new alpha-(1,6) glycosidic linkages. Subsequently, it produces isomaltose, panose, isomaltotriose, and panose series oligosaccharides. The favorite acceptor of the enzyme is maltose, which generates panose and a free glucose monosaccharide after the transfer reaction. If glucose accumulates, it also acts as a glycosyl-acceptor to produce isomaltose and consequently produces isomaltose series oligosaccharides. After terminating the activity of enzymes, the accumulated glucose and some of the remaining maltose are removed by yeast fermentation and/or with a filtration system. After downstream purification steps such as decolorization, filtration, and ion exchange system, the final product consists of 90% (w/w) on a dry basis of isomalto-oligosaccharides (DPs from 2 to 13), also called IMO900, which we call commercial IMO here.

Commercial IMO is manufactured by several companies globally. The chemical composition of commercial IMO varies with the source of starch, the commercial enzymes to use, the enzymatic processing, and downstream processing. For example, VitaFiber® IMO, produced by BioNeutra North America Inc. based in Alberta, Canada, is available in syrup and powder in commercial quantities and is sold to customers across the globe. On average, the main components of commercial IMO are isomaltose (average 20% (w/w) on dry basis), panose (depends on different products), and isomaltotriose (around 8-10% on dry basis), and higher oligomers with DPs more than 4. VitaFiber® IMO tastes sweet and shows around 40-60% of the sweetness of sucrose. The DE value is approximately 32-35, which also approximates the relative sweetness of the product. Maltodextrin (DEs less than 20) does not taste sweet. VitaFiber® IMO is recognized as a generally regarded safe (GRAS) ingredient by the U.S.-FDA and is approved as a novel food ingredient by the European Commission and Health Canada. It is not only lower in calories than regular sugar, but also it is a natural source of prebiotic fiber. The dietary fiber content of commercial IMO products depends on the manufacturing processing and the commercial enzymes used. VitaFiber® IMO is significantly less digestible than maltose or maltodextrin and more digestible than RD. IMO may promote health by modulating intestinal microbiota depending on their DPs and ratio of alpha-(1,4) to alpha-(1,6) linkages.

### SUMMARY

The method described herein may be used as an alternative to aims to modify the conventional biocatalytic strategy of IMO to produce AGO, which increases the slow or non-digestible linkages to increase the fiber content. The AGO product also maintains some level of sweetness, which is similar to IMO. The enzymatic process that produces AGO may include three steps. The first step is liquefaction. The second is a transfer reaction with enzymes such as glycosyltransferases, starch branching enzyme, cyclodextrin glucanotransferase, etc. The third step is a secondary transfer reaction with transglucosidase with or without additional saccharification of glucoamylase. The second and third steps are the sequential or simultaneous process of saccharification and transfer reactions. Compared with the current commercial IMO process, the present invention method comprises the method to prepare AGO by enzymatic bioconversion based on starch and also include transglucosidase reaction. Compared to commercial IMO, the AGO product has an increased proportion of non- or slowly-digestible linkages to 10-30%, increased average molecular weight by 100-300, and increased fiber content to 40-70%. The sugar content was decreased to 15-22%.

The chemical structure of the AGO produced is different from the commercial IMO, with slightly higher molecular weight and different glycosidic linkages to enhance the fiber content for the benefit of human health. The DE value of the AGO product is between 20-30 and remains similar in sweetness to commercial IMO which is higher than the sweetness of maltodextrins but lower than that of sucrose. The product AGO may be used as functional-health oligosaccharides in food, beverage, feed, etc. It is also may be used in cosmetic or pharmaceutical products.

The method described herein may be used as an alternative to the manufacturing method of IMO to produce AGO, which not only increases the slow or non-digestible linkages but also maintains some level of sweetness. The enzyme-catalysis process produces AGO with two or more transfer reactions, including transglucosidase, glycosyltransferases, starch branching enzyme, cyclodextrin glucanotransferase with transferase activity, and a sequential or simultaneous process of saccharification and transfer reactions. The method may be implemented to provide a preparation method for AGO with enhanced fiber content. The method uses enzymatic bioconversion of starch to produce AGO, integrating two or more transfer reactions to generate more digestion-resistant linkages and maintain similar sweetness than commercial IMO product.

According to an aspect, a method for preparing AGO includes the following steps:
a) Preparing a liquefied starch solution having a mass concentration of 20 to 45% (w/v) with a DE between 7 and 15, and a pH value of 5.5-8.0.
b) Contacting the solution with one or more transferases selected from the group consisting of starch branching enzyme, cyclodextrin glucanotransferase, Glyco Transferase, or transglucosidase into the solution in the presence or absence of alpha-amylase or beta-amylase;
c) Incubating the solution at about 50-65° C. for 16-48 hours, terminating the reaction by raising the temperature of the solution to 90-100° C. for 20-30 minutes, followed by cooling the solution to 50-60° C., and adjusting the pH value of the reaction solution to 5.0-6.0.
d) Contacting the solution with transglucosidase in the presence or absence of alpha-amylase or beta-amylase and incubating for about 24-58 hours;
e) Terminating the enzymes by raising the temperature of the solution to 90-100° C. for 20-30 minutes, or contacting the solution with glucoamylase and incubating for about 1-8 hours before terminating the enzymes.
f) Processing the downstream product after one or more transferases reactions or sequential transglucosidase reaction and saccharification, which includes unit operations such as yeast fermentation, membrane filtration system or chromatography separation removing monosaccharides and disaccharides, decoloring and filtration, ion exchange to desalt and remove proteins, and concentration under vacuum to a solid content concentration of at least 50% (w/v) by mass.

The liquefied starch solution may be prepared by conventional liquefication similar to the typical commercially scaled processing for producing starch-derived IMO. In one example, one part of starch may be mixed with 1-3 parts of water and then mixed with 0.01-0.04% (w/w) of CaCl₂) based on the dried starch to get the homogenous starch slurry at a final concentration of 10-22° Bé. One or two liquefaction enzymes may be mixed with the starch slurry in an amount of 3600-36000 U per millilitre of starch slurry with 20-45% (w/v), after the pH value was adjusted to 5.5-7.0 with hydrochloric acid, citric acid, sodium carbonate, or sodium hydroxy. The homogenous mixture with thermostable alpha-amylase may be liquefied through a jet liquefier at a temperature of around 100° C. and held at around 90° C. until the solution passes the Iodine test and achieves a DE value of 7-15. The reaction may be terminated by steam at around 130° C. Without a jet liquefier, the homogenous starch slurry with alpha-amylase of 3600-36000 U per millilitre of starch slurry may be kept warm with vigorous agitation at 70-90° C. for 15-60 mins. When the Iodine test reaches the required value, the DE value may be measured over time. When the DE value reaches 7-15, the reaction may be terminated at 90-100° C. for 20-30 minutes.

According to an aspect, there is provided a method for producing AGO using starch as the raw material utilizing the biocatalytic technology of starch-modified enzymes, modifying and transferring the oligosaccharides or polysaccharides molecules by two or more transfer reactions to form non- or slow-digestible linkage types in a sequential or simultaneous processing of saccharification and transfer reactions. The additional saccharification by glucoamylase is another option after transfer reactions to increase fiber content in the product. The three steps of the enzyme-catalysis process may include liquefaction, the first stage of transfer reaction, and the second stage of transfer reaction with a sequential or simultaneous process of saccharification. The present method may comprise enzymatic bioconversion based on starch and common transglucosidase reactions. Various environmental factors may affect the rate of enzyme-catalyzed reactions through reversible or irreversible changes in protein structure. The effects of pH, temperature, enzyme-substrate rate, and reaction time are generally well understood. Normally, enzymes gradually lose activity over time. If the commercially available enzymes are used and stored in proper cool and dry conditions, the enzyme activity and properties may be maintained well before the expiration day, such as about 94-97% during 52 weeks or about 95-88% of residual enzymatic activity may be expected depending on storage time and temperature. The enzyme dose may be based on the dried starch. Another factor may include the interaction of saccharification enzymes and transferases on the substrates, which the former mainly produces suitable substrates for the latter.

According to an aspect, the composition of the AGO product may have an increased proportion of non- or slowly-digestible linkages of 50-75%, an increased average molecular weight by 100-300, and an increased fiber content to 40-65%. AGO. The sugar content was decreased to 15-25% on dry basis while largely maintaining the sweetness of the product. It results in the decrease of DE to 20-30 and maintaining similar sweetness to commercial IMO products. In contrast, commercial IMO typically contains predominantly alpha-(1,6)-glycosidic linkages, which are slowly digestible, digestible alpha-(1,4)-glycosidic linkages, and a small portion of other linkage types such as alpha-(1,2)- and alpha-(1,3)-glycosidic linkages which are resistant to digestion.

According to another aspect, the produced AGO may be used as a general food ingredient or novel food in a number of food categories, including beverages, cereal products, sugar confectionery, nutritionally complete and fortified foods. The AGO may provide a sweet taste and rheological attributes as the sweetener in food and beverages. The AGO may use as a functional oligosaccharide product or ingredient in food, beverage, feed, cosmetic, or pharmaceutical products.

According to an aspect, there is provided a method for producing alpha-glucooligosaccharides (AGO), comprising the steps of: providing a source of starch; processing the source of starch to produce a liquefied starch solution with a mass concentration of between 10 to 45% (w/v) and a Dextrose Equivalent (DE) of between 7 to 15; adjusting the pH value of the liquefied starch solution to between 5.5-8.0; contacting the liquefied starch solution with one or more transfer reaction enzymes and incubating the liquefied starch solution at a temperature of between 55-65° C. for between 16-48 hours to produce a reaction solution; terminating the transfer reaction enzymes within the reaction solution; adjusting the pH value of the reaction solution to 5.0-6.0; contacting the reaction solution with transglucosidase and incubating the reaction solution for 24-56 hours; terminating the transglucosidase; and producing an AGO product.

According to other aspects, the method may include one or more of the following features, alone or in combination: the liquefied starch slurry may be produced by contacting the source of starch with a thermostable alpha-amylase in a jet liquefaction process, or with an alpha-amylase in a heating process such that the liquefied starch slurry has a DE of between 7-15; the transfer reaction enzyme may comprise a starch branching enzyme, cyclodextrin glucosyltransferase, Glyco Transferase, transglucosidase, or combinations thereof; the transfer reaction enzyme may comprise Glyco Transferase in an amount of 0.9-80 U per millilitre of liquefied starch solution or reaction solution; the transfer reaction enzyme may comprise a starch branching enzyme in an amount of 15-375 U per millilitre of liquefied starch solution or reaction solution; the transfer reaction enzyme may comprises cyclodextrin glucosyltransferase in an amount of 0.5-15 U per millilitre of liquefied starch solution or reaction solution; the transfer reaction enzyme may comprise transglucosidase in an amount of 270-2700 U per millilitre of liquefied starch solution or reaction solution; the transfer reaction enzyme may comprise two or more enzymes selected from a group consisting of: bacterial alpha-amylase in an amount of 72-1440 U per millilitre of liquefied starch solution, fungal alpha-amylase in an amount of 0.2-4.8 U per millilitre of liquefied starch solution, and beta-amylase in an amount of 0.2-4.8 U per millilitre of liquefied starch solution; the liquefied starch solution may be contacted with two or more transfer reaction enzymes that react sequentially with the liquefied starch solution; the liquefied starch solution may be contacted with two or more transfer reaction enzymes that react simultaneously with the liquefied starch solution; incubating the liquefied starch solution may comprise saccharification by contacting the liquefied starch solution with glucoamylase in an amount of 0.1-1.5 U per millilitre of liquefied starch solution for between 1-8 hours; the source of starch may comprises tapioca starch, tapioca flour, pea starch, pea flour, corn starch, corn flour, potato starch, potato flour, rice starch, rice flour, wheat starch, wheat flour, and combinations thereof; producing the AGO product may comprise downstream processing that comprises yeast fermentation, membrane filtration, chromatographic separation to remove monosaccharides and disaccharides, decoloring and filtration, ion exchange to desalt and remove proteins, concentration under vacuum to a solid content of at least 50% by mass, or combinations thereof, the AGO product may be suitable for use in food, beverage, feed, cosmetic, or pharmaceutical products after the downstream processing; the AGO may be water-soluble, a degree of polymerization (DPs) of the AGO product may be substantially between 2 and 20, and wherein less than 25% w/w of the AGO product has a DP of greater than 10; the AGO may be a low molecular alpha-glucan that comprises non-digestible or slow-digestible linkage types, wherein between 5-30% w/w of linkages are alpha-(1,3) linkages and between 30-65% of the linkages are alpha-(1,6) linkages; the Dextrose Equivalent of the AGO product may be between 20-30; the AGO may have a sweetness that is higher than that of maltodextrins and lower than that of sucrose; the AGO may have a water-soluble dietary fiber contend of at least 40% as measured by AOAC method 2009.01.

According to an aspect, there is provided an alpha-glucooligosaccharides (AGO) product, produced by the method described above.

According to an aspect, there is provided a carbohydrate composition comprising between 1-99 wt % (dry solids basis) of AGO composition produced by the method described above.

### BRIEF DESCRIPTION OF THE DRAWINGS

These and other features will become more apparent from the following description in which reference is made to the appended drawings, the drawings are for the purpose of illustration only and are not intended to be in any way limiting, wherein:

FIG. 1 is a flowchart of a manufacturing method for IMO.

FIG. 2 is a flowchart of a manufacturing method for AGO.

### DETAILED DESCRIPTION OF PREFERRED EMBODIMENTS

There will now be described a method of producing AGO using biocatalytic processing.

As used herein, “about” will be understood by persons of ordinary skill in the art and may vary depending upon the context in which it is used, such as a relatively small amount of variation that permits the intended result to be achieved. If there are uses of the term which are not clear to persons of ordinary skill in the art, given the context in which it is used, “about” may mean up to, plus, or minus 5% of the particular term.

When a range of values is provided herein, the range is intended to be inclusive of the upper and lower numbers in the range. Where appropriate, the defined range is intended to include immaterial variations of the top and bottom that provide similar results.

The term “dietary fiber” or “fiber” herein may refer to non-digestible carbohydrates or polysaccharides that are intrinsic to plant cell walls, however, in the broad sense, may include low-digestible water-soluble polysaccharides and non-digestible oligosaccharides, which are not digested by human digestion system. This definition may be considered in light of other definitions, such as the Codex Alimentarius, which defines fiber as carbohydrate polymers with ten or more monomers that are not hydrolyzed by enzymes in the small intestine of humans. Purified and synthetic carbohydrates may be included in the definition if they have been shown to have a physiological effect or benefit to health. This definition is used by regulatory agencies for approval of fiber-related health claims; many jurisdictions include oligosaccharides with a degree of polymerization of 3 or higher in the definition of fiber. This definition is used by regulatory agencies for approval of fiber-related health claims; oligosaccharides with a degree of polymerization of 3 or higher are often included in the definition of fiber. In addition, Health Canada defined “dietary fiber” in 2012 as referring to carbohydrates with a DP of 3 or more that are not digested and absorbed by the small intestine; it includes traditional fibers, which naturally occur in foods of plant origin, and novel fibers that synthetically produced or are obtained from natural sources and have at least one fiber physiological effect demonstrated by generally accepted scientific evidence.

The term “disaccharide” herein may refer to a carbohydrate that consists of two monosaccharide units linked by a glycosidic linkage. The term “oligosaccharide” herein may refer to a carbohydrate that consists of 3 to 10 monosaccharide units linked by glycosidic linkages, and may include up to 20 monosaccharide units or even disaccharides. An oligosaccharide may also be referred to herein as an “oligomer”.

The term “isomalto-oligosaccharides”, “IMO”, or “IMOs” herein may refer to isomalto-oligosaccharides preparations, which are generally accepted as a mixture of glucosyl oligosaccharides with both alpha-(1,6)- and alpha-(1,4)-linkages together with small proportions of alpha-(1,3)- or alpha-(1,2)-linkages, even though it inherently means a mixture of glucosyl oligosaccharides only containing alpha-(1,6)-linkages. The main degrees of polymerization of IMO are from DP 2 to 6. The main components include isomaltose, panose and isopanose, isomaltotriose, isomaltotetraose, and their higher oligosaccharides. Isomaltose, maltose, and panose would be digested in the small intestine and absorbed as glucose following oral administration. The slowly- or non-digested fractions of IMO would consist of the larger oligosaccharides and pass through the small intestine and undergo microbial fermentation as fully fermentable oligosaccharides in the large intestine. IMO occur naturally in honey and fermented foods, such as miso, sake, and soy sauce. IMO has been widely used as an alternative sweetener in foods and beverages.

The term “alpha-glucooligosaccharides”, also called “alpha-gluco-oligosaccharides”, may be abbreviated “AGO” herein and may refer to a mixture of oligosaccharides of D-glucose monomers linked with alpha-glucosidic linkages. To this extent, IMO may be considered to belong to AGO. Relative to IMO, AGO may have increased alpha-(1,6)-linkages, alpha-(1,3)-linkages, alpha-(1,4)-linkages, and/or cyclic alpha-1,4-linkages, and small proportions of alpha-(1,2)-linkages. It may also be called alpha-glucan oligosaccharides (GOS). Alpha-glucans are polysaccharides and homopolymers that may include glucose monomers joined by alpha-glucosidic linkages, mainly alpha-(1,2), alpha-(1,3), alpha-(1,4), and alpha-(1,6) linkages. GOS are shorter chain (lower DPs) composed of same D-glucose monomers linked through these alpha-glucosidic linkages. Starch is one of the most commonly studied alpha-glucans. The main degrees of polymerization of AGO are from DP 2 to 15 and it may have a DP lower than 20. AGO may be used as a sweetener product and a replacement for IMOs in foods and beverages.

Based on the reaction mechanism, substrate specificity, and protein sequence, the commercially available microbial enzymes used for starch modification or conversion have been classified into glucosyl hydrolases (EC3.2.1.X) which only catalyse the hydrolysis of glycosidic bonds and glycosyltransferases (EC2.4.X.Y) which can catalyse both the hydrolysis of glycosidic bonds and the formation of new glycosidic bonds.

The terms “glycosidic linkage” and “glycosidic bond” may be used interchangeably to refer to the type of covalent bond that joins a carbohydrate molecule to another carbohydrate molecule. The term “alpha-(1,3)-glycosidic linkage” may refer to an alpha-(1,3)-glycosidic linkage between two alpha-D-glucose units. The terms “alpha-(1,6)-glycosidic linkage” may refer to an alpha-(1,6)-glycosidic linkage between two alpha-D-glucose units. The term “alpha-(1,4)-glycosidic linkage” may refer to an alpha-(1,4)-glycosidic linkage between two alpha-D-glucose units.

The term “liquefaction” herein may refer to a process of partial enzymatic hydrolysis of granular starch slurry into starch hydrolysate with different DEs. The process may use thermostable amylases and high temperature to convert the highly viscous gelatinized starch into liquid dextrin or oligosaccharides.

The term “liquefaction enzyme” may refer to an enzyme such as alpha-amylase used for liquefaction processing. The term “alpha-amylase” may refer to an alpha-(1,4)-glucan-4-glucanohydrolase (EC 3.2.1.1) that catalyzes the hydrolysis of internal alpha-(1,4)-glycosidic linkages in large molecular weight polysaccharides, such as starch which yields shorter chains with an alpha-configuration, dextrins, and oligosaccharides, which constitute a mixture of maltose, maltotriose, and branched oligosaccharides of 6-8 glucose units that contain both alpha-(1,4) and alpha-(1,6) linkages. Alpha-amylase may be used in the conversion of starch into oligosaccharides. The bacterial alpha-amylase may be derived from Bacillus spp. such as Bacillus licheniformis, Bacillus amyloliquefaciens, or Bacillus stearothermophilus. It is commercially available such as Termamyl® 2X, Termamyl® SC DS, BAN® 480 L, and LpHera® from Novozymes, such as SEBstar HTL from Enzyme Innovation, as Kleistase SD80 from AMANO enzyme.

The term “saccharification” may refer to the process of breaking an oligosaccharide into its monosaccharide, disaccharide, or short chain oligosaccharides components. The term “saccharification enzyme” refers to suitable enzymes for saccharification and may include alpha-amylase, fungal alpha-amylase, beta-amylase, and glucoamylase. Many of the bacterial alpha-amylase mentioned as above for liquefaction enzymes to break down the starch molecules may be used for saccharification to hydrolyze the long chain oligosaccharides into shorter chain oligosaccharides. The fungal alpha-amylase derived from Aspergillus oryzae is commercially available such as Fungamyl® 800 L and Maltera® Standard from Novozymes. The term “beta-amylase” herein refers to 4-alpha-D-glucan maltohydrolase (EC 3.2.1.2), which are exoenzymes that cleave the penultimate alpha-(1,4) linkage from the nonreducing end of the polymeric chains and release beta-maltose by an inversion. The term “beta” relates to the initial anomeric configuration of the free sugar group released and not to the configuration of the linkage hydrolyzed. Beta-amylase derived from Bacillus flexus is commercially available such as Secura® from Novozymes and beta-Amylase F “Amano” from Amano Enzyme Inc.

The term “glucoamylase” may refer to exo-acting enzymes (EC 3.2.1.3) that are generally recognized that catalyze the hydrolysis of both alpha-(1,4) and alpha-(1,6)-linked glycosidic linkages from non-reducing ends of glucose-containing oligo- and polysaccharides. The glucoamylase derived from Aspergillus niger is commercially available such as AMG®, AMYLASE AG®, Dextrozyme®, and Extenda® from Novozymes, and as SEBamyl GL from Enzyme Innovation.

The term “debranching enzyme” may refer to pullulanase (EC 3.2.1.41), a starch-debranching enzyme in the α-amylase family, specifically cleaves alpha-1,6-glycosidic linkages in starch-type polysaccharides, such as pullulan, alpha-limit dextrin, glycogen, and amylopectin. It has been widely utilized in debranching and hydrolyzing starch completely. The pullulanase expressed from a genetically modified Bacillus subtilis is commercially available such as Promozyme® D2 and Promozyme® D6 from Novozymes.

The term “alpha-glucosidase” may refer to enzymes (EC 3.2.1.20) that catalyze the hydrolytic release of alpha-D-glucose from the non-reducing ends of alpha-(1,4)-linked oligosaccharide and polysaccharide substrates. They typically show diverse substrate specificities. Alpha-glucosidases may also have hydrolytic activity toward alpha-(1,3) and alpha-(1,6)-linked glucosyl-glucose linkages. Some retaining alpha-glucosidases from Aspergillus niger, Bacillus stearothermophilus, etc., exhibit high and significant transglycosylation activity. In this description, the term “transglucosidase” may refer to the alpha-glucosidase from Aspergillus niger which can efficiently catalyze transglycosylation as a crucial enzyme to produce commercial IMO. The term “transglycosylation” may refer to the reaction in that transglucosidase catalyzes both hydrolytic and transfer reactions on incubation with certain alpha-D-glucose-oligosaccharides converting an alpha-(1,4)-linkage to an alpha-(1,6)-linkages. It can also transfer a glucosyl residue to form kojibiose or nigerose at a very lower level. The transglucosidase derived from Aspergillus niger is commercially available such as Transglucosidase L “Amano” from Amano Enzyme and Transglucosidase from Danisco-Genencor.

The term “transfer reaction” may include the glycosyltransfer reactions catalyzed by glycosyltransferases, glucanotransferases, and transglucosidase, and the term “transglycosylation” herein refers to the reactions catalyzed by transglucosidase, a kind of alpha-glucosidase with transglycosylation activity.

The term “glycosyltransferase” may refer to a ubiquitous group of enzymes that catalyze the transfer of glycosyl and sugar moieties from activated donor molecules onto specific acceptor molecules including saccharide acceptors forming glycosidic bonds or non-saccharide acceptors. They may display diversity in their donor, acceptor, and product specificity and thereby generate a potentially infinite number of glycoconjugates, oligo- and polysaccharides.

The term “Glyco Transferase” is used as a trade name of the enzyme “Glyco Transferase ‘Amano’ L” derived from Aeribacillus pallidus (previously identified as Geobacillus pallidus) strain manufactured by Amano Enzyme Inc. It is a 4-alpha-glucanotransferase (EC 2.4.1.25) or so-called maltotriose transferase enzyme. It can act on soluble starch and its hydrolysates, including maltodextrin and maltooligosaccharides on alpha-(1,4) glucoside linkages to transfer maltotriose units to oligo- and polysaccharides with alpha-(1,3) glucoside linkages. It acts as a special trisaccharide transfer reaction.

The term “starch branching enzyme” used herein is an alpha-1,4-glucan 6-glycosyltransferase (EC 2.4.1.18) which catalyzes the hydrolysis of alpha-1,4 linked linear chains followed by glycosyltransferase action to create new alpha-1,6 linked branch chains. The conversion of linear starch molecules amylose into amylopectin or highly branched structures may increase their solubility and accessibility for glycosyl hydrolases. The starch branching enzyme used herein is a branching enzyme derived from Rhodothermus obamensis commercially available such as Branchzyme® from Novozymes.

The term “cyclodextrin glycosyltransferase” or “cyclodextrin glucanotransferases” may refer to enzymes that have intramolecular transglycosylation reaction to produce cyclic alpha-1,4 linked oligosaccharides typically containing 6, 7, and 8 glucopyranose units from starch, called alpha-, beta- and gamma-cyclodextrins, respectively. Besides cyclization, these enzymes may also catalyze intermolecular transglycosylation via coupling and disproportionation. The cyclodextrin glycosyltransferase (EC 2.4.1.19) derived from Thermoanaerobacter sp. is commercially available such as Toruzyme® 3.0 L from Novozymes and derived from Geobacillus sp. or CGT-SL from Amano Enzyme. Toruzyme® 3.0 L mainly catalyzes the formation of alpha-, beta- and gamma-cyclodextrins from starch. The term “cyclodextrin” may refer to alpha-, beta- and gamma-cyclodextrins which are glucooligosaccharides with a special cyclic alpha-1,4 linkage bonds. They are generally recognized as safe (GRAS) and are widely applied in food industries with a hydrophilic surface and a hydrophobic central cavity for the solubilization and stabilization of hydrophobic colorant and fragrance compounds. CAVAMAX® W6 alpha-cyclodextrin manufactured by Wacher Chemie are marketed as a non-digestible and fully fermentable dietary fiber using in novel food-supplement formulations and the European Commission has certified that alpha-cyclodextrin has a proven health claim. Beta-cyclodextrin, similar to alpha-cyclodextrin, is not digested in the upper gastrointestinal tract but is fermented by the large intestinal microflora. Gamma-cyclodextrin is more readily digested by amylases than alpha- and beta-cyclodextrin, but only less than 10% of gamma-cyclodextrin among all cyclodextrins is produced by Toruzyme® 3.0 L and CGT-SL.

An “immobilized” enzyme may refer to an enzyme that is attached to inert, insoluble material. The term “molecular weight” of glucose oligomers or short chains alpha-glucan may be represented as number-average molecular weight (Mn) or as weight-average molecular weight (Mw). Alternatively, the molecular weight can be represented as Daltons, grams/mole.

The term “yeast fermentation” may refer to the use Saccharomyces cerevisiae yeast cells applied after the final step of biosynthesis to eliminate undesirable sugars such as glucose, maltose, and maltotriose to improve further purity of obtained IMO or AGO.

The term “dextrin” or “maltodextrin” may refer to starch hydrates after liquefaction with a DE value of less than 10.

The terms “increased”, “enhanced,” and “improved” may be used interchangeably and may refer to a greater quantity or activity, such as a quantity or activity slightly greater than the original quantity or activity or a quantity or activity in large excess compared to the original quantity or activity and including all quantities or activities in between.

The terms “weight-weight percentage” or “% (w/w)” and the like may be used interchangeably herein. As for a composition, mixture, or other solution, the weight-weight percentage (% w/w) on a dry basis refers to the percentage of the dry matter.

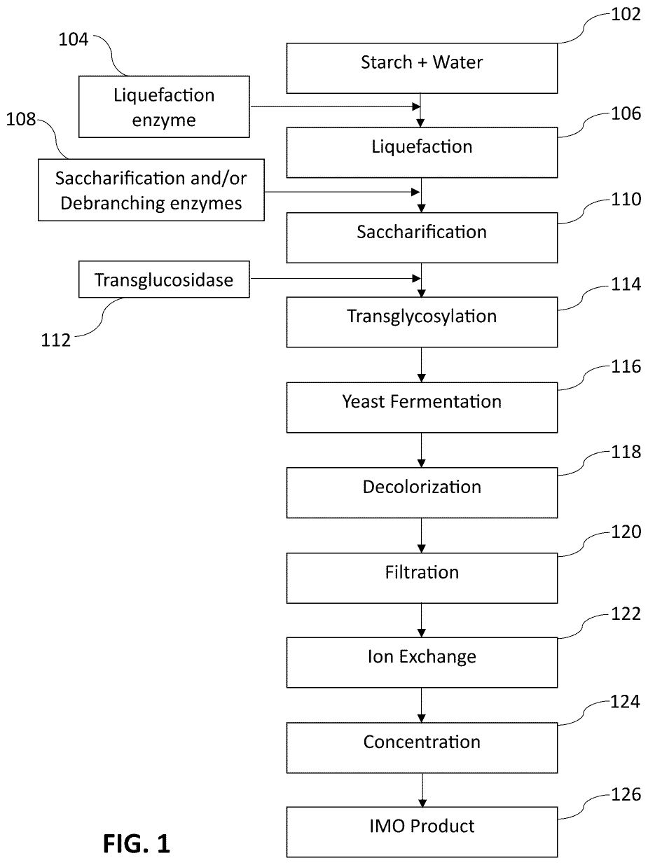

Referring to FIG. 1, a method of producing IMO is shown. In step 102, a starch/water slurry is provided. In step 104, a liquefaction enzyme is added and in step 106, the slurry is liquefied. In step 108, saccharification and/or debranching enzymes are added and in step 110, the mixture is conditioned to cause a saccharification reaction to occur. In step 112, transglucosidase is added and in step 114, the mixture is conditioned to cause a transglycosylation reaction to occur. Thereafter, additional processing steps may be applied to generate a product suitable for the intended use. Examples of such steps may include a yeast fermentation in step 116 to remove a substantial portion of the accumulated glucose, a decolorization step 118, a filtration step 120, an ion exchange step 122, and a concentration step 124 to produce a final IMO product 126.

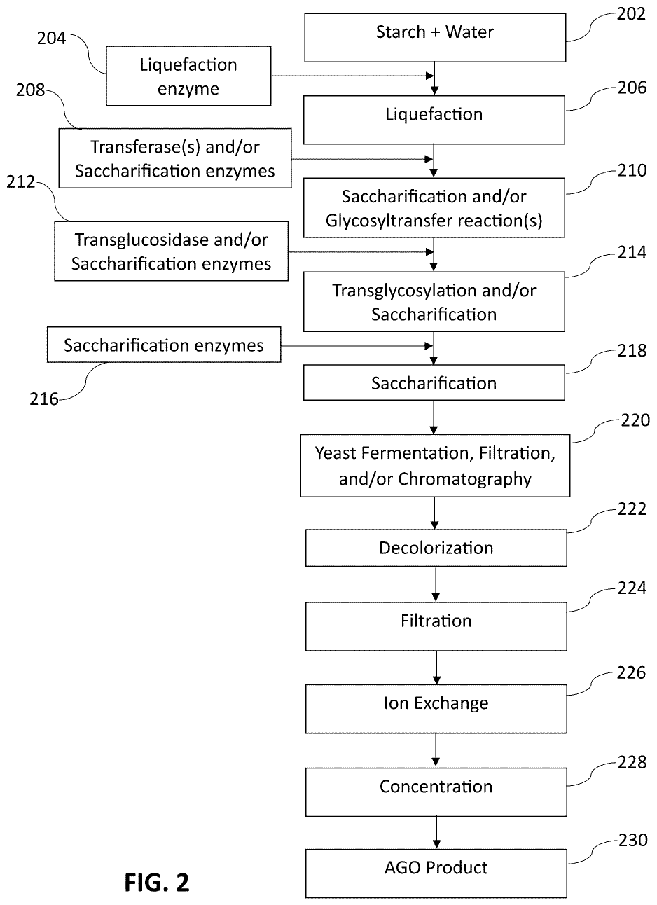

Referring to FIG. 2, an example of a process to produce AGO is shown. While this differs from the process to produce IMO, some of the reactions are similar and may be adapted into the process used to produce AGO. Referring to step 202, the process starts by providing a starch slurry, a liquefaction enzyme is provided in step 204, and the slurry is liquefied in step 206. These steps may be similar to the liquefaction process discussed above and may be similar to that used for manufacturing IMO. The discussion below will focus primarily on the enzymatic reactions using liquefied starch solutions as the starting materials between the liquefaction and downstream purification processes. The process steps include adding transferase and/or saccharification enzymes in step 208 and processing the mixture to achieve desired saccharification and/or glycosyltransfer reaction(s) in step 210. In step 212, transglucosidase and/or saccharification enzymes are added and in step 214, the mixture is reacted to achieve transglycosylation and/or saccharification. In step 216, further saccharification enzymes are added and in step 218, a further saccharification reaction is achieved. Suitable enzymes and mixture conditions, such as temperature, pH, and reaction time, may be as discussed elsewhere. The mixture is then subjected to further downstream processing, which may be similar to the steps discussed with respect to FIG. 1. This may include yeast fermentation and/or filtration 220, or other suitable steps such as chromatography separation to remove a substantial portion of the accumulated glucose and/or disaccharides. Other downstream steps may follow the general processes, such as decoloring 222, filtration 224, ion exchange 226 to deionize and remove proteins, concentration 228 such as by vacuum distillation to a solid content concentration of at least 50° Bx to achieve the AGO product 230. These downstream process steps may be varied according to known practices based on the intended use of the final product.

In the processes described above, suitable sources of starch may include one or more of the following: tapioca starch, tapioca flour, pea starch, pea flour, corn starch, corn flour, potato starch, potato flour, rice starch, rice flour, wheat starch, and wheat flour.

Specific examples of various processes that may be used to produce the desired results will be discussed below. It will be understood that the various steps may be combined other than in the manner discussed. For example, different reactions may be combined together to produce AGO with a preferred linkage content. As such, the reactions described below are intended to describe specific examples within a range of alternatives that permit variations in the final product, and those skilled in the art will understand that the product may be adjusted by combining different aspects presented in the examples discussed below.

In some implementations, oligosaccharide production may include sequential saccharification and transglycosylation, in which a liquefied starch solution prepared as mentioned above is kept at 50-60° C. with the pH adjusted to 5.0-6.0, and then subsequently subjected to the saccharification reaction by contacting with a solution of bacterial alpha-amylase at the amount of 72-1440 U, or fungal alpha-amylase or beta-amylase at the amount of 0.2-4.8 U and a solution of pullulanase at the amount of 0.12-2.4 U per millilitre of liquefied starch solution for 2-16 hours. The enzymes may be deactivated by heating at 90-100° C. for 20-30 minutes. The solution may be contacted with transglucosidase in the amount of 270-2700 U per millilitre of reaction solution and kept at 50-60° C. for 24-58 hours. The enzymes may be deactivated by heating at 90-100° C. for 20-30 minutes. Using these steps, the final product may include alpha-1,6 linkages of more than 50%, while the rest are mainly linked by alpha-1,4 linkages, which is much higher than alpha-1,3 or alpha-1,2 linkages.

In some processes, oligosaccharide production may include simultaneous saccharification and transglycosylation, in which a liquefied starch solution prepared as mentioned above is kept at 50-60° C. with the pH adjusted to 5.0-6.0, and then contacted with a solution of bacterial alpha-amylase at the amount of 72-1440 U, or fungal alpha-amylase or beta-amylase at the amount of 0.2-4.8 U and a solution of transglucosidase at the amount of 270-2700 U per millilitre of liquefied starch solution and kept at for 24-58 hours. The enzymes were deactivated by heating at 90-100° C. for 20-30 minutes.

In some processes, the enzyme termination using high temperature between reactions may be omitted so that the sequential reactions occur simultaneously instead.

In some processes, oligosaccharide production may include simultaneous saccharification and transglycosylation, and then secondary saccharification. The liquefied starch solution prepared as mentioned above may be kept at 50-60° C., with the pH adjusted to 5.0-6.0, and contacted with a solution of transglucosidase in the amount of 270-2700 U and bacterial alpha-amylase at the amount of 72-1440 U, or fungal alpha-amylase or beta-amylase at the amount of 0.2-4.8 U per millilitre of liquefied starch solution for 24-58 hours. The solution may be reacted with a solution of enzyme comprising glucoamylase at the amount of 0.1-1.5 U per millilitre of reaction solution and kept at around 50-60° C. for 1-8 hours. The enzymes may be deactivated by heating at 90-100° C. for 20-30 minutes.

In some processes, AGO may be produced using secondary saccharification after two or three transfer reactions. After two or three transfer reactions, the solution may be reacted with glucoamylase in the amount of 0.1-1.5 U per millilitre of reaction solution and kept at around 50-60° C. for 1-8 hours. The enzymes may be deactivated by heating at 90-100° C. for 20-30 minutes.

In some processes, after enzymatic reactions of IMO or AGO, the glucose may be removed by yeast fermentation, followed by decoloring, filtration, and ion exchange and then concentration. In some examples, the glucose or even maltose and isomaltose may be removed by chromatography separation. After chromatography separation, the fractions with DP more than 2 were collected. In some endorsements, the fraction with low molecular weight, such as monosaccharides and disaccharides, were separated from the fraction with high molecular weight by the filtration system, including one or more weight cut-off membrane elements.

In some processes, AGO may be produced using simultaneous saccharification and transglycosylation followed by secondary transglycosylation. The liquefied starch solution prepared as mentioned above may be kept at 50-60° C., the pH adjusted to 5.0-6.0, and contacted with a solution of transglucosidase in the amount of 270-2700 U and a solution of bacterial alpha-amylase at the amount of 72-1440 U, or fungal alpha-amylase or beta-amylase at the amount of 0.2-4.8 U per millilitre of liquefied starch solution for 16-56 hours. The enzymes may be deactivated by heating at 90-100° C. for 20-30 minutes. After removal of accumulated glucose by yeast fermentation, filtration system or chromatography separation, the pH of the solution may be adjusted to 5.0 and then reacted with a solution of transglucosidase in the amount of 270-2700 U per millilitre of reaction solution and kept at around 50-60° C. for 24-58 hours. The enzyme may be deactivated by heating at 90-100° C. for 20-30 minutes.

In some processes, AGO may be produced using first and second transfer reactions including simultaneous Glyco Transferase reaction and saccharification, and sequential transglycosylation. The liquefied starch solution prepared as mentioned above may be kept at 55-65° C. with the pH adjusted to 6.5-8.0 and contacted with a solution of Glyco Transferase in the amount of 0.9-90 U and a solution of bacterial alpha-amylase at the amount of 72-1440 U, or fungal alpha-amylase or beta-amylase at the amount of 0.2-4.8 U per millilitre of liquefied starch solution for 16-48 hours. The enzymes may be deactivated by heating at 90-100° C. for 20-30 minutes. The pH value of the solution may then be adjusted to 5.0-6.0. The solution may then be contacted with transglucosidase in the amount of 270-2700 U per millilitre of reaction solution at around 50-60° C. for 24-56 hours, and the enzyme was deactivated by heating at 90-100° C. for 20-30 minutes.

In some processes, AGO may be produced using first and second transfer reactions including sequential Glyco Transferase reaction, and simultaneous saccharification and transglycosylation. The liquefied starch solution prepared as mentioned above may be kept at 55-65° C. with the pH adjusted to 6.5-8.0 and contacted with a solution of Glyco Transferase in the amount of 0.9-90 U per millilitre of liquefied starch solution for 16-48 hours. The enzyme may be deactivated by heating at 90-100° C. for 20-30 minutes. The pH value of the solution may be adjusted to 5.0-6.0 and reacted with transglucosidase in the amount of 270-2700 U and bacterial alpha-amylase at the amount of 72-1440 U or fungal alpha-amylase or beta-amylase at the amount of 0.2-4.8 U per millilitre of reaction solution and maintained at around 50-60° C. for 24-56 hours. The enzymes may be deactivated by heating at approximately 90-100° C. for 20-30 minutes. This may result in the final AGO product having alpha-1,3 linkages of about 15-25%, and alpha-(1,6) linkages of about 10-15% among other alpha-glucosidic linkages.

In some processes, AGO may be produced using first and second transfer reactions, including partially simultaneous saccharification and Glyco Transferase reaction and sequential transglycosylation. The liquefied starch solution prepared as mentioned above may be kept at 55-65° C. with the pH adjusted to 6.0-8.0 and contacted with a solution of bacterial alpha-amylase at the amount of 72-1440 U or fungal alpha-amylase or beta-amylase at the amount of 0.2-4.8 U per millilitre of liquefied starch solution for 1-4 hours, and then a solution of Glyco Transferase in the amount of 0.9-90 U per millilitre of reaction solution may be contacted for 16-48 hours at 55-65° C. The enzymes may be deactivated by heating at 90-100° C. for 20-30 minutes. The pH value of the solution may be adjusted to 5.0-6.0 and reacted with transglucosidase in the amount of 270-2700 U per millilitre of reaction solution and kept at around 50-60° C. for 16-56 hours. The enzyme may be deactivated by heating at approximately 90-100° C. for 20-30 minutes.

In some processes, AGO may be produced by first and second transfer reactions including sequential Glyco Transferase reaction, simultaneous saccharification and transglycosylation, and secondary saccharification. The liquefied starch solution prepared as mentioned above may be kept at 55-65° C. with the pH adjusted to 6.5-8.0 and contacted with a solution of Glyco Transferase in the amount of 0.9-90 U per millilitre of liquefied starch solution for 16-48 hours. The enzyme may be deactivated by heating at 90-100° C. for 20-30 minutes. The pH value of the solution may be adjusted to 5.0-6.0 and reacted with transglucosidase in the amount of 270-2700 U and bacterial alpha-amylase at the amount of 72-1440 U or fungal alpha-amylase or beta-amylase at the amount of 0.2-4.8 U per millilitre of reaction solution and keep at around 50-60° C. for 16-56 hours. The solution may be reacted with enzyme comprises glucoamylase at the amount of 0.1-1.5 U per millilitre of reaction solution and kept at around 50-60° C. for 1-8 hours. The enzymes may be deactivated by heating at approximately 90-100° C. for 20-30 minutes.

In some processes, AGO may be produced by first and second transfer reactions including sequential starch branching enzyme reaction and simultaneous saccharification and transglycosylation. The liquefied starch solution prepared as mentioned above may be kept at 55-65° C. the final AGO product to 5.5-6.5 and contacted with a solution of starch branching enzyme in the amount of 15-375 U per millilitre of liquefied starch solution for 12-24 hours. The enzyme may be deactivated by heating at 90-100° C. for 20-30 minutes. The pH value of the solution may be adjusted to 5.0-6.0 and reacted with transglucosidase in the amount of 270-2700 U and bacterial alpha-amylase at the amount of 72-1440 U or fungal alpha-amylase or beta-amylase at the amount of 0.2-4.8 U per millilitre of reaction solution and kept at around 50-60° C. for 24-56 hours. The enzymes may be deactivated by heating at approximately 90-100° C. for 20-30 minutes.

In some processes, AGO may be produced by three transfer reactions including simultaneous starch branching enzyme and Glyco Transferase reactions, and simultaneous saccharification and transglycosylation. The liquefied starch solution prepared as mentioned above may be kept at 55-65° C. with the pH adjusted to 6.0-7.0. A solution of starch branching enzyme in the amount of 15-375 U per millilitre of liquefied starch solution and a solution of Glyco Transferase in the amount of 0.9-90 U per millilitre of liquefied starch solution may be contacted for 16-48 hours. The enzymes may be deactivated by heating at 90-100° C. for 20-30 minutes. The pH of the solution may be adjusted to 5.0-6.0 and reacted with transglucosidase in the amount of 270-2700 U and bacterial alpha-amylase at the amount of 72-1440 U or fungal alpha-amylase or beta-amylase at the amount of 0.2-4.8 U per millilitre of reaction solution and maintained at around 50-60° C. for 24-56 hours. The enzymes may be deactivated by heating at approximately 90-100° C. for 20-30 minutes.

In some processes, AGO may be produced by three transfer reactions including sequential cyclodextrin glucanotransferase reaction and Glyco Transferase reaction, and simultaneous saccharification and transglycosylation. The liquefied starch solution may be prepared as mentioned above may be kept at 55-65° C. with the pH adjusted to 5.5-6.5. A solution of cyclodextrin glucanotransferase in the amount of 0.5-15 U per millilitre of liquefied starch solution may be contacted for 12-24 hours, after which the enzyme may be deactivated by heating at 90-100° C. for 20-30 minutes. The pH value of the solution may be adjusted to 6.5-8.0. A solution of Glyco Transferase in the amount of 0.9-90 U per millilitre of reaction solution may then be added and the mixture may be incubated at 55-65° C. for 16-48 hours. The enzyme may be deactivated by heating at 90-100° C. for 20-30 minutes. The pH value of the solution may be adjusted to 5.0-6.0 and reacted with transglucosidase in the amount of 270-2700 U and bacterial alpha-amylase at the amount of 72-1440 U or fungal alpha-amylase or beta-amylase at the amount of 0.2-4.8 U per millilitre of reaction solution and maintain at around 50-60° C. for 16-48 hours. The enzymes may then be deactivated by heating at approximately 90-100° C. for 20-30 minutes.

In some processes, AGO may be produced by first, second, and third transfer reactions, including simultaneous cyclodextrin glucanotransferase and Glyco Transferase reactions, and simultaneous saccharification and transglycosylation. The liquefied starch solution may be prepared as mentioned above and kept at 55-65° C. with the pH adjusted to 6.5-7.5. A solution of cyclodextrin glucanotransferase in the amount of 0.5-15 U per millilitre of liquefied starch solution and a solution of Glyco Transferase in the amount of 0.9-90 U per millilitre of liquefied starch solution may then be added and the mixture incubated for 16-48 hours. The enzymes may then be deactivated by heating at 90-100° C. for 20-30 minutes. The pH value of the solution may be adjusted to 5.0-6.0 and reacted with transglucosidase in the amount of 270-2700 U and fungal or bacterial alpha-amylase or beta-amylase in the amount of 270-2700 U and maintained at around 50-60° C. for 16-56 hours. The enzymes may then be deactivated by heating at approximately 90-100° C. for 20-30 minutes.

### EXAMPLES

The present technology is further illustrated by the following non-limiting examples. The Examples described below illustrate methods for producing and manufacturing AGO that pertain to the present technology. The downstream processing may be undertaken following the general processes to obtain the final AGO product.

The following enzymes, which are used in the examples below, are commercially available were stored in proper cool and dry conditions. As such, the enzymes can be expected to be fully or mostly active before the expiration day.
a) Fungamyl® 800 L (Fungamyl®) is a fungal alpha-amylase obtained from a selected strain of Aspergillus oryzae, commercially available from Novozymes with an enzyme activity of 800 FAU/g. One Fungal alpha-Amylase Unit (FAU) is the amount of enzyme which breaks down 5.26 g starch per hour at Novozymes' standard method for determination of alpha-amylase. See the Analytical Method for further information. It maintains its declared activity for 6 months when stored in closed container at 0-10° C.
b) Transglucosidase L “Amano” (Transglucosidase) derived from Aspergillus niger strain is an alpha-glucosidase, commercially available from Amano Enzyme with a transglucosidase activity of 300 KU/ml.
c) BAN® 480 L (BAN®) is a bacterial alpha-amylase derived from Bacillus amyloliquefaciens, commercially available from Novozymes with an enzyme activity of 480 KUN-B/g.
d) Dextrozyme® DX 1.5× (Dextrozyme®) is a mixture of glucoamylase derived from Aspergillus niger and pullulanase produced by a genetically modified strain of Bacillus, commercially available from Novozymes with a glucoamylase activity of 255 AGU/g and a pullulanase activity of 510 NPUN/g.
e) Termamyl® SC DS (Termamyl®) containing thermostable alpha-amylase that hydrolyzes (1,4)-alpha-D-glucosidic linkages in starch polysaccharides with an enzymatic activity of 240 KNU-S/g.
f) Beta-Amylase F “Amano” (Beta-amylase) is a beta-amylase manufactured by a submerged fermentation process derived from Bacillus flexus, commercially available from Amano Enzyme Inc. with an enzyme activity of 650 U/g.
g) Glyco Transferase “Amano” L (Glyco Transferase) derived from Aeribacillus pallidus (previously identified as Geobacillus pallidus) strain is a 4-alpha-glucanotransferase (EC 2.4.1.25) or so-called maltotriose transferase enzyme with an enzyme activity of 3000 U/mL, commercially available from Amano Enzyme Inc. One unit of activity is defined as the quantity of enzyme that liberates 1 umol of glucose per minute using maltotetraose as substrate at pH 6.5 and 40° C. for 60 min.
h) Branchzyme® is a glycosyltransferase also called branching enzyme that hydrolyzes alpha-1,4 linkages in starch to create new side chains with alpha-1,6 linkages with an enzyme activity of 25000 BEU/g. The activity of branching glycosyltransferase is determined by measuring the rate of the amount of enzyme which transfers a segment of alpha-(1,4)-alpha-D-glucan chain to a primary hydroxy group in a similar glucan chain to create 1,6-alpha-linkages. The activity of branching glycosyltransferase is determined by measuring the rate of introduction of 1,6-alpha-linkages into the substrate amylose. One branching enzyme unit (BEU) is defined as the quantity of the enzyme that causes a decrease in absorbance at 660 nm of an amylose-iodine complex of 1% per minute under standard conditions at pH 7.2 and 60° C.
i) CGT-SL is a cyclodextrin glucanotransferase derived from Geobacillus sp. with an enzyme activity of 400 U/mL, commercially available from Amano Enzyme. Toruzyme® 3.0 L (Toruzyme®) derived from Thermoanaerobacter sp. is a cyclodextrin glucanotransferase an enzyme activity of 3 KNU-CP/g, commercially available from Novozymes.
j) Transglucosidase L “Amano” (Transglucosidase) is a transglucosidase derived from Aspergillus niger with a transglucosidase activity of 300,000 U/mL, commercially available from Amano Enzyme.

### Example 1

The liquefied starch solution was prepared as described above with a DE value of 9 and was kept at 60° C. with the pH adjusted to 5.5 and contacted with a solution of Transglucosidase in the amount of 900 U and a solution of Fungamyl® in the amount of 1.2 U per millilitre of liquefied starch solution for 24 hours. The enzymes were deactivated by heating at around 100° C. for 20 minutes. After removal of accumulated glucose by yeast fermentation, filtration system, or chromatography separation, the pH value of the solution was adjusted to 5.0 and the solution was reacted with a solution of Transglucosidase in the amount of 900 U per millilitre of reaction solution and kept at around 55° C. for 48 hours. The enzymes were deactivated by heating at around 95° C. for 20 minutes.

### Example 2

The liquefied starch solution was prepared as described above with a DE value of 11 and was kept at 65° C. with the pH adjusted to 8.0 and contacted with a solution of Glyco Transferase in the amount of 9 U per millilitre of liquefied starch solution was contacted for about 20 hours. The enzyme was deactivated by heating at 95° C. for 20 minutes. The pH value of the solution was adjusted to 5.0 and contacted with a solution of Transglucosidase in the amount of 2700 U per millilitre of reaction solution and a solution of Fungamyl® in the amount of 2.4 U per millilitre of reaction solution and kept at around 60° C. for 48 hours. The enzymes were deactivated by heating at approximately 95° C. for 20 minutes.

### Example 3

The liquefied starch solution was prepared as described above with a DE value of 12 and was kept at 65° C. with the pH adjusted to 6.5 and contacted with a solution of Glyco Transferase in the amount of 9 U and a solution of Termamyl® in the amount of 720 U per millilitre of liquefied starch solution for 24 hours. The enzymes were deactivated by heating at around 100° C. for 20 minutes. The pH value of the solution was adjusted to 5.5. The solution was contacted with a solution of Transglucosidase in the amount of 450 U per millilitre of reaction solution at 60° C. for 48 hours, and the enzyme was deactivated by heating at around 100° C. for 20 minutes.

### Example 4

The liquefied starch solution was prepared as described above with a DE value of 7 and was kept at 60° C. with the pH adjusted to 8.0 and contacted with a solution of BAN in the amount of 1440 U per millilitre of liquefied starch solution and kept at around 60° C. for 4 hours, and then a solution of Glyco Transferase in the amount of 18 U per millilitre of reaction solution was contacted for 48 hours. The enzyme was deactivated by heating at around 95° C. for 20 minutes. The pH value of the solution was adjusted to 5.5. The solution was contacted with a solution of Transglucosidase in the amount of 1800 per millilitre of reaction solution and kept at about 55° C. for 48 hours. The enzymes were deactivated by heating at approximately 95° C. for 20 minutes.

### Example 5

The liquefied starch solution was prepared as described above with a DE value of 10 and was kept at 65° C. with the pH adjusted to 6.5 and contacted with a solution of Glyco Transferase in the amount of 9 U and a solution of Fungamyl® in the amount of 1.2 U per millilitre of liquefied starch solution for 24 hours. The enzymes were deactivated by heating at around 95° C. for 20 minutes. The pH value of the solution was adjusted to about 5.0. The solution was contacted with a solution of Transglucosidase in the amount of 450 U per millilitre of reaction solution and kept at about 55° C. for 48 hours, and the enzyme was deactivated by heating at around 95° C. for 20 minutes.

### Example 6

The liquefied starch solution was prepared as described above with a DE value of 9 and was kept at 65° C. with the pH adjusted to 6.5 and contacted with a solution of Glyco Transferase in the amount of 9 U and a solution of Fungamyl® in the amount of 0.2 U per millilitre of liquefied starch solution for 24 hours. The enzymes were deactivated by heating at around 95° C. for 20 minutes. The pH value of the solution was adjusted to 6.0. The solution was contacted with a solution of Transglucosidase in the amount of 450 U per millilitre of reaction solution and kept at around 50° C. for 56 hours. The enzymes were deactivated by heating at around 95° C. for 20 minutes. The solution was contacted with a solution of Dextrozyme® in the amount of 0.5 U per millilitre of reaction solution and kept at around 50° C. for 4 hours. The enzymes were deactivated by heating at approximately 95° C. for 20 minutes.

### Example 7

The liquefied starch solution was prepared as described above with a DE value of 9 and was kept at 60° C. with the pH adjusted to 6.5. A solution of Branchzyme® in the amount of 225 U and a solution of Glyco Transferase in the amount of 4.5 U per millilitre of liquefied starch solution was added and contacted for 24 hours. The enzyme was deactivated by heating at around 95° C. for 20 minutes. The pH value of the solution was adjusted to 6.0 and reacted with a solution of Transglucosidase in the amount of 2700 U and a solution of Beta-amylase in the amount of 3.9 U per millilitre of reaction solution and kept at around 55° C. for 48 hours. The enzymes were deactivated by heating at approximately 95° C. for 20 minutes.

### Example 8

The liquefied starch solution was prepared as described above with a DE value of 7 and was kept at 70° C. with the pH adjusted to 6.5. A solution of Toruzyme® in the amount of 9 U per millilitre of liquefied starch solution based on dried starch was contacted for 16 h, and the enzyme was deactivated by heating at around 95° C. for 20 minutes. A solution of Glyco Transferase in the amount of 4.5 U per millilitre of reaction solution was added and the mixture was incubated at 55° C. and pH 6.5 for 48 hours. The enzyme was deactivated by heating at around 90° C. for 20 minutes. The pH value of the solution was adjusted to 5.5 and reacted with a solution of Transglucosidase in the amount of 900 U and a solution of Fungamyl® of 0.7 U per millilitre of reaction solution based on dried starch and kept at around 60° C. for 48 hours. The enzymes were deactivated by heating at approximately 95° C. for 20 minutes.

### Example 9

The liquefied starch solution was prepared as mentioned above with a DE value of 13 and was kept at 60° C. with the pH adjusted to 6.5. A solution of CGT-SL in the amount of 1.2 U and a solution of Glyco Transferase in the amount of 4.5 U per millilitre of liquefied starch solution were added and the mixture was incubated for 48 hours. The enzymes were deactivated by heating at around 90° C. for 20 minutes. The pH value of the solution was adjusted to 5.5 and reacted with a solution of Transglucosidase in the amount of 900 U and a solution of Fungamyl® and kept at around 55° C. for 48 hours. The enzymes were deactivated by heating at approximately 90° C. for 30 minutes.

### Results

Select results from the examples described above are shown in Table 1 below.

TABLE 1
The Dietary fiber contents, carbohydrate composition
and DE values of AGO products (Data on dry basis)

| Example | | Glucose | Maltose | Isomaltose | Panose | Isomaltotriose | Dietary fiber | |
|---|---|---|---|---|---|---|---|---|
| # | Product | (%) | (%) | (%) | (%) | (%) | (%) ^{a} | DE |
| 1 | AGO | 1.86 | 4.61 | 14.26 | 7.04 | 11.16 | 44.3 | 24 |
| 2 | AGO | 1.31 | 3.62 | 13.96 | 6.83 | 7.25 | 44.1^{b} | 27 |
| 3 | AGO | <1.00 | 2.41 | 13.57 | 3.51 | 6.13 | 48.4 | 25 |
| 8 | AGO | <1.00 | 3.26 | 23.3 | 3.71 | 9.78 | 52.2 | 29 |
| 9 | AGO | <1.00 | 2.02 | 16.91 | 3.11 | 6.36 | 61.5 | 27 |

^{a} Dietary fiber contents were measured with the in-house total dietary fiber test with a Megazyme kit based on AOAC Method 2009.01 and 2011.25.
^{b}The dietary fiber content was tested by a third-party laboratory with the AOAC Method 2009.01.

### Methods of Measurement

Below are descriptions of certain methods of measurement used in the description above to determine the amounts measured above. Other suitable measurement methods may be used, which may lead to slight variations in the measured results.

### Carbohydrate Profile

The contents of glucose, maltose, isomaltose, panose and isomaltotriose were quantitatively analysed using an Agilent 1200 Series high-performance liquid chromatography (HPLC) with 70% acetonitrile mobile phase using the same component as the external standard.

### Determination of Dextrose Equivalent (DE) by Lane-Eynon Titration

The reducing sugar content in samples was determined based on the reduction of cupric (II) sulfate in an alkaline tartrate solution. The Standardization of Fehling mixture solutions was carried out by titrating a mixture of 12.5 mL of Fehling A and 12.5 mL of Fehling B with a glucose standard solution at 6% (w/v). The concentration of copper (II) sulfate in Fehling A was adjusted so that the amount of cupric (II) sulfate was sufficient to react with 20.0±1.0 mL of standard glucose solution. The sample weight was calculated as the following formula based on the estimated reducing sugars content with the anticipated DE. The sample solution was dropped at 18 mL and mixed with Fehling's mixture A and B in a conical flask and gently boiled for 2 minutes. Two drops of methylene blue indicator were added, and the sample was added dropwise until the blue color in the flask completely disappeared. The titration after boiling was completed within 1 minute.

### Glycosidic Bond Features

A small amount of sample was dissolved in D₂O to make 0.7 ml of solution and added to 5 mm NMR tube. A Presat sequence with a 30-degree pulse and a total relaxation delay of 5.2 s was used with a 600 MHz NMR spectrometer to obtain the ¹H-NMR spectra of the sample.

### Total Solid Content (TTS)

The solid content (%) of the samples was determined by using Milwaukee MA871 Digital Refractometer. The reading was displayed as % Brix, commonly used to measure dissolved solid content (such as sucrose) of an aqueous solution. The degrees Brix (° Bx) is calculated by dividing the mass of the solid substances by the total mass of the liquid sample multiplied by 100.

### Dietary Fibre Analysis

The dietary fiber (DF) of the samples was measured using Megazyme Total Dietary Kit (K-INTDF 04/20) based on AOAC Method 2009.01 and 2011.25.

### Iodine Test

Put approximately 5-6 drops of the sample by micropipette in a 25-mL beaker. Add approximately 2-3 mL of distilled water. Add 1 drop of iodine solution (5% w/v), then mix well, and observe the colour. Amylose is a linear but not a straight chain of glucose units but instead is coiled like a spring, with six glucose monomers per helix that can accommodate an iodine molecule to form the amylose-iodine complex in characteristic blue-violet colour. The helical structure of amylopectin is disrupted by the branching of the chain, so instead of the deep blue-violet colour amylose gives with iodine, amylopectin produces a less intense reddish brown. In amylopectin, the branching points are clustered and allow the formation of allomorphs. The vicinal glucan chains thereby form double helices. The double helices are formed by neighboring alpha-1,4-linked glucan chains with a degree of polymerization (DP) from 10 to 20.

In this patent document, the word “comprising” is used in its non-limiting sense to mean that items following the word are included, but items not specifically mentioned are not excluded. A reference to an element by the indefinite article “a” does not exclude the possibility that more than one of the elements is present, unless the context clearly requires that there be one and only one of the elements.

The scope of the following claims should not be limited by the preferred embodiments set forth in the examples above and in the drawings, but should be given the broadest interpretation consistent with the description as a whole.

1. A method for producing alpha-glucooligosaccharides (AGO), comprising the steps of:
providing a source of starch;
processing the source of starch to produce a liquefied starch solution with a mass concentration of between 10 to 45% (w/v) and a Dextrose Equivalent (DE) of between 7 to 15;
adjusting a pH value of the liquefied starch solution to between 5.5-8.0;
contacting the liquefied starch solution with one or more transfer reaction enzymes and incubating the liquefied starch solution at a temperature of between 55-65° C. for between 16-48 hours to produce a reaction solution;
terminating the transfer reaction enzymes within the reaction solution;
adjusting a pH value of the reaction solution to between 5.0-6.0;
contacting the reaction solution with transglucosidase and incubating the reaction solution for between 24-56 hours;
terminating the transglucosidase; and
producing an AGO product.

2. The method of claim 1, wherein the liquefied starch solution is produced by contacting the source of starch with a thermostable alpha-amylase in a jet liquefaction process, or with an alpha-amylase in a heating process such that the liquefied starch solution has a DE of between 7-15.

3. The method of claim 1, wherein the transfer reaction enzyme comprises a starch branching enzyme, cyclodextrin glucosyltransferase, Glyco Transferase, transglucosidase, or combinations thereof.

4. The method of claim 1, wherein the transfer reaction enzyme comprises Glyco Transferase in an amount of between 0.9-80 U/mL of liquefied starch solution or reaction solution.

5. The method of claim 1, wherein the transfer reaction enzyme comprises a starch branching enzyme in an amount of between 15-375 U/mL of liquefied starch solution or reaction solution.

6. The method of claim 1, wherein the transfer reaction enzyme comprises cyclodextrin glucosyltransferase in an amount of between 0.5-15 U/mL of liquefied starch solution or reaction solution.

7. The method of claim 1, wherein the transfer reaction enzyme comprises transglucosidase in an amount of between 270-2700 U/mL of liquefied starch solution or reaction solution.

8. The method of claim 1, wherein the transfer reaction enzyme comprises two or more enzymes selected from a group consisting of: bacterial alpha-amylase in an amount of between 72-1440 U/mL of liquefied starch solution, fungal alpha-amylase in an amount of between 0.2-4.8 U/mL of liquefied starch solution, and beta-amylase in an amount of between 0.2-4.8 U/mL of liquefied starch solution.

9. The method of claim 1, wherein the liquefied starch solution is contacted with two or more transfer reaction enzymes that react sequentially with the liquefied starch solution.

10. The method of claim 1, wherein the liquefied starch solution is contacted with two or more transfer reaction enzymes that react simultaneously with the liquefied starch solution.

11. The method of claim 1, wherein incubating the liquefied starch solution comprises saccharification by contacting the liquefied starch solution with glucoamylase in an amount of between 0.1-1.5 U/mL of liquefied starch solution for between 1-8 hours.

12. The method of claim 1, wherein the source of starch comprises tapioca starch, tapioca flour, pea starch, pea flour, corn starch, corn flour, potato starch, potato flour, rice starch, rice flour, wheat starch, wheat flour, and combinations thereof.

13. The method of claim 1, wherein producing the AGO product comprises downstream processing that comprises yeast fermentation, membrane filtration, chromatographic separation to remove monosaccharides and disaccharides, decoloring and filtration, ion exchange to desalt and remove proteins, concentration under vacuum to a solid content of at least 50% by mass, or combinations thereof.

14. The method of claim 13, wherein the AGO product is suitable for use in food, beverage, feed, cosmetic, or pharmaceutical products after the downstream processing.

15. The method of claim 1, wherein the AGO is water-soluble.

16. The method of claim 1, wherein a degree of polymerization (DPs) of the AGO product is substantially between 2 and 20, and wherein less than 25% w/w of the AGO product has a DP of greater than 10.

17. The method of claim 1, wherein the AGO is a low molecular alpha-glucan that comprises non-digestible or slow-digestible linkage types, wherein between 5-30% of linkages are alpha-(1,3) linkages and between 30-65% of the linkages are alpha-(1,6) linkages.

18. The method of claim 1, wherein the Dextrose Equivalent of the AGO product is between 20-30.

19. The method of claim 1, where the AGO has a sweetness that is higher than that of maltodextrins and lower than that of sucrose.

20. The method of claim 1, wherein the AGO has a water-soluble dietary fiber contend of at least 40% as measured by AOAC method 2009.01.

21. An alpha-glucooligosaccharides (AGO) product, produced by the method of claim 1.

22. A carbohydrate composition comprising between 1-99 wt % (dry solids basis) of AGO composition produced by the method of claim 1.
